(19) United States
(12) Patent Application Publication
(10) Pub. No.: US 2005/0261603 A1
(43) Pub. Date: Nov. 24, 2005

(54) Transparent biopsy punch

(76) Inventor: Gregory P. Wittenberg, Rapid City, SD (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/849,757

(22) Filed: May 20, 2004

(51) Int. Cl. A61B 17/34

(52) U.S. Cl. 600/566

(57) ABSTRACT

A skin biopsy punch has a transparent cutting edge, allowing a user to view a patient's skin in and around a portion to be cut. In one embodiment, the cutting edge allows viewing of a skin lesion and the lesion's margins. By viewing the margins, the lesion may be cleanly removed or excised with a higher rate of negative or clear surgical margins.

### FIELD OF THE INVENTION

The present invention relates to biopsy punches, and in particular to a transparent biopsy punch.

### BACKGROUND OF THE INVENTION

A skin biopsy punch is a device which samples a piece of skin. Biopsy punches have taken many different forms, with common punches having a cylindrical metal cutting blade that is circular or elliptical in shape. The blades may have a beveled cutting edge. Some punches are formed with an aperture or window positioned over the top of the cutting edge, allowing one to look down inside the blade to view tissue as it is being cut. However, these devices do not allow convenient simultaneous viewing of the tissue at or outside the cutting edge.

### SUMMARY OF THE INVENTION

A skin biopsy punch has a transparent cutting edge, allowing a user to view a patient's skin in and around a portion to be cut. In one embodiment, the cutting edge allows viewing of a skin lesion and the lesion's margins. By viewing the margins, the lesion may be cleanly removed or excised with a higher rate of negative or clear surgical margins.

The cutting edge is formed of a material that is clear, and in one embodiment, has little or no distortion, such as glass or polycarbonate. In a further embodiment, a handle coupled to the cutting edge is also transparent, and the combination may be formed in one piece. The handle may extend straight out from the cutting edge to provide optimal leverage, or may extend at an angle.

### BRIEF DESCRIPTION OF THE DRAWINGS

FIG. 1 is a cross section view of a biopsy punch according to an example embodiment of the invention.

FIG. 2 is a bottom view of the biopsy punch of FIG. 1 from a cutting edge end of the biopsy punch according to an example embodiment of the invention.

FIG. 3 is a partial cross section view an alternative biopsy punch according to an example embodiment of the invention.

FIG. 4 is a top view of an alternative biopsy punch according to an example embodiment of the invention.

FIG. 5 is a partial cross section view of a further alternative biopsy punch according to an example embodiment of the invention.

FIG. 6 is a bottom view of an alternative biopsy punch according to an example embodiment of the invention.

### DETAILED DESCRIPTION OF THE INVENTION

In the following description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Dimensions shown in the drawings may be exaggerated to more clearly show certain aspects, and such dimensions should not be taken as limiting of the scope of the invention. The following description is, therefore, not to be taken in a limited sense, and the scope of the present invention is defined by the appended claims.

FIG. 1 shows a single piece biopsy punch generally at 100. Punch 100 has a handle 110 for gripping and manipulating the punch by hand or with other devices as desired. The handle 110 tapers into a cutting section 115 which is generally smaller in diameter than the handle 110 in some embodiments. The cutting section 115 tapers into a cutting edge indicated at 120. The cutting edge is sharp, to provide a means to take a biopsy of a skin lesion. Section 115 contains an exterior bevel on its distal end, ending in the cutting edge 120.

The cutting edge 120 may be used to sample a piece of skin or remove specific skin lesions in their entirety. The transparent nature of the punch or tool allows for visualization of the skin lesion including the lesion's margins. By viewing the margins, the lesion may be clearly removed or excised with a high rate of negative or clear surgical margins. A higher percentage of negative margins may result in better cure rates.

In one embodiment, the cutting section 115 and cutting edge 120 is formed of a material that is substantially clear, with little or no distortion. The material may be glass or plastic, such as a polycarbonate type material. The cutting edge 120 is sharp, beveled, and non-brittle in one embodiment to provide clean skin samples without debris. In one embodiment, the cutting edge 120 is as thin as possible to provide a sharp edge, yet strong enough to avoid chipping.

The cutting edge may be sharpened, or formed in an injection mold. In one embodiment, the entire biopsy punch 100 is formed of a single piece of clear or transparent material. Section 115 and cutting edge 120 in one embodiment, is sufficiently long and sufficiently transparent to allow acceptable viewing of the area of skin to be cut, such as the lesion.

Other materials that may be used for the cutting edge 120, and cutting section 115 include polyvinyl chloride, styrene acrylonitrile, acrylonitrile styrene, unsaturated polyester, allylics, epoxies and vinyl esters. These plastics are rigid and translucent. Other materials may also be used, such as diamond, either mined or fabricated.

In one embodiment, the handle 110 is pencil shaped to be grasped easily between two or three fingers. A shelf 125 between the cutting edge and the handle 110 may be formed to prevent too deep a biopsy by providing a positive stop. The internal diameter of the cutting edge 120 may be any width desired, and is commonly between approximately 1 mm to 4 mms. While shown as circular in shape, the cutting edge may have different shapes, such as elliptical or even polygonal.

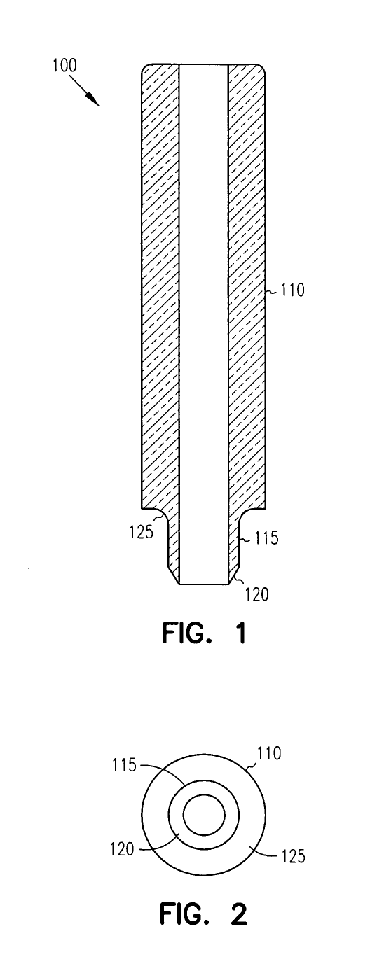

A bottom view of the biopsy punch 100 is shown in FIG. 2.

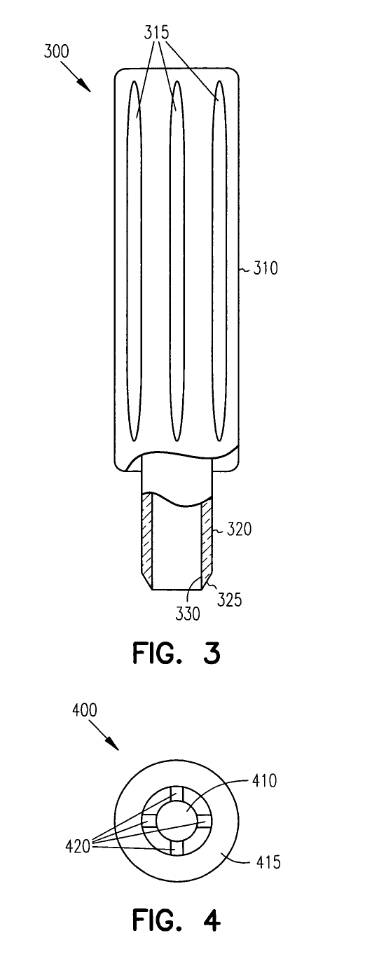

FIG. 3 shows an alternative biopsy punch 300 with a portion of it shown in cross section. Biopsy punch 300 comprises a handle 310 with gripping structures 315, such as ribs or grooves to facilitate gripping by a hand or other instrument. In this embodiment, the handle 310 is formed an a separate material from a cutting section 320, which is coaxially fixed at one end of the handle 310 such as by glue, heat, friction fit or other means. The handle may be transparent if desired, or made of any type of material that provides sufficient support for the cutting section, such as wood, plastic, metal, or the same material as the cutting section. The handle 310 may be formed in many different shapes, such as generally round, elliptical, triangular, polygonal, or any other ergonomic type of shape if desired. Cutting section 320 is beveled as indicated at 325, and ends in a sharp cutting edge 330.

FIG. 4 is a top view of yet a further alternative biopsy punch at 400. A cutting section 410 is coupled to a handle 415 by means of supports 420. Four supports 420 are shown in this embodiment, but other numbers of supports may be utilized as desired. The supports may be glued or other wise adhered to support the cutting section 410 in a desired relationship to the handle 415.

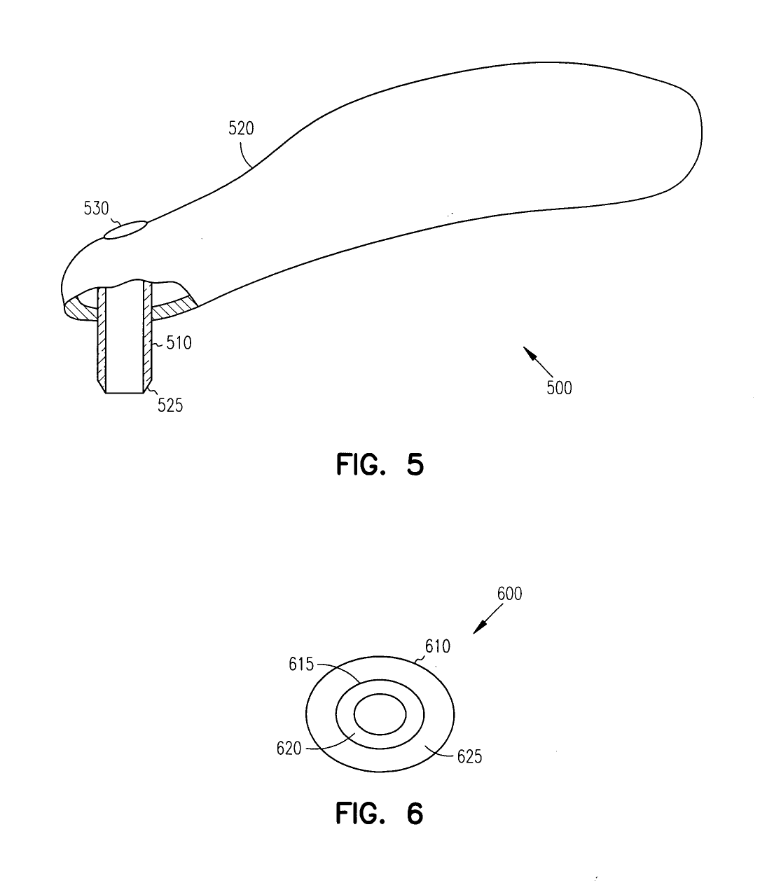

FIG. 5 is a partial cross section view of a further alternative biopsy punch 500 according to an example embodiment of the invention. In this embodiment, a cutting section 510 is supported by a handle 520 that extends laterally away from the cutting section at an angle. Handle 520 in one embodiment, is shaped to fit in a hand, and the angle may be varied as desired to provide adequate usability for taking skin samples from various positions on a body. The shape of the handle may be varied as a function of desired ergonomics, and may come in different sizes for different sized hands. Tapered handles are used in one embodiment.

In one embodiment, the cutting section 510 has a first biopsy cutting edge 525, and a second end 530 extending through the handle 520, allowing viewing of the skin being sampled through the second end, and also through the transparent cutting section 510. The handle may also be transparent, opaque, or partially transparent about the cutting section 510 to enhance viewability.

FIG. 6 is a bottom representation of the shape of an alternative biopsy punch 600. Punch 600 has a handle 610 for gripping and manipulating the punch by hand or with other devices as desired. The handle 610 transitions into a cutting section 615 which is generally smaller in diameter than the handle 610 and is elliptical in shape. In some embodiments, other shapes may be utilized, such as polygonal shapes that may be designed for specific types of desired incisions. The cutting section 615 tapers into a cutting edge indicated at 620. The cutting edge is sharp, to provide a means to take a biopsy of a skin lesion. Cutting section 615 contains an exterior bevel on its distal end, ending in the cutting edge 620.

When using one of the above biopsy punches to take a skin sample, a health care professional, such as doctor will place a biopsy punch proximate an area of skin to be sampled, such as just above the area. The skin to be sampled is then viewed through a transparent cutting section of the biopsy punch, allowing positioning of the biopsy punch based on such viewing. At this point, a biopsy of the skin may be taken in a normal manner.

1. A biopsy punch comprising:
a handle;
a transparent cutting section; and
a transparent cutting end of the cutting section providing visibility of skin being sampled by the biopsy punch.

2. The biopsy punch of claim 1 and further comprising a shelf proximate the cutting end to provide a positive stop.

3. The biopsy punch of claim 1 wherein the handle is transparent.

4. The biopsy punch of claim 1 wherein the cutting section is formed of a transparent plastic.

5. The biopsy punch of claim 4 wherein the cutting section is formed of polycarbonate.

6. The biopsy punch of claim 1 wherein the cutting section is formed of glass.

7. The biopsy punch of claim 1 and further comprising gripping structures on the handle.

8. The biopsy punch of claim 1 wherein the cutting section is hollow, allowing viewing of the skin being sampled through the middle of the cutting section.

9. The biopsy punch of claim 1 wherein the cutting section has a chamfer on the cutting end that provides a sharp cutting edge.

10. The biopsy punch of claim 1 wherein the cutting edge is a shape selected from the group consisting of circular, elliptical and polygonal.

11. A biopsy punch comprising:
a transparent cutting section;
a handle fixed to the cutting section and extending laterally away from the cutting section at a desired angle; and
a transparent cutting end of the cutting section, wherein the cutting section extends from the handle to provide visibility of skin being sampled by the biopsy punch.

12. The biopsy punch of claim 11 wherein the cutting section is hollow, and the handle allows viewing of the skin through the hollow cutting section.

13. The biopsy punch of claim 12 wherein the cutting section extends through the handle.

14. The biopsy punch of claim 11 and further comprising a shelf proximate the cutting end to provide a positive stop.

15. The biopsy punch of claim 11 wherein at least a portion of the handle is transparent.

16. The biopsy punch of claim 11 wherein the cutting section is formed of a transparent plastic.

17. The biopsy punch of claim 16 wherein the cutting section is formed of polycarbonate.

18. The biopsy punch of claim 11 wherein the cutting section is formed of glass.

19. The biopsy punch of claim 9 and further comprising gripping structures on the handle.

20. The biopsy punch of claim 11 wherein the cutting section has a chamfer on the cutting end that provides a sharp cutting edge.

21. A method of taking a biopsy of skin, the method comprising:
placing a biopsy punch proximate an area of skin to be sampled;
viewing the skin to be sampled through a transparent cutting section of the biopsy punch;
positioning the biopsy punch based on such viewing; and
taking a biopsy of the skin.
